(19) United States
(12) Patent Application Publication
(10) Pub. No.: US 2025/0288510 A1
(43) Pub. Date: Sep. 18, 2025

(54) Makeup Composition Comprising a Polyphenol, a Polyoxyalkylenated or Polyglycerolated Compound, a Monoalcohol and an Acid, and Method Employing Same

(71) Applicant: L'OREAL, Paris (FR)

(72) Inventors: Marine GUITTOT, Paris (FR); Roshanak DEBEAUD, Chevilly Larue (FR); Boudiaf BOUSSOUIRA, Chevilly Larue (FR)

(73) Assignee: L'OREAL, Paris (FR)

(21) Appl. No.: 18/860,152

(22) PCT Filed: Apr. 24, 2023
(86) PCT No.: PCT/EP2023/060575

§ 371 Date: Oct. 25, 2024

(30) Foreign Application Priority Data
Apr. 28, 2022 (FR) FR2204039
Jun. 15, 2022 (FR) FR2205847

(51) Int. Cl. A61K 8/60; A61K 8/34; A61Q 1/04

(52) U.S. Cl. CPC A61K 8/604; A61K 8/34; A61K 8/347; A61Q 1/04

(57) ABSTRACT

The invention relates to a composition for making up and/or caring for the skin and/or the lips, preferably the lips, more particularly a makeup composition, comprising a) at least one polyphenol X comprising at least two different phenol groups; b) at least one compound Y chosen from those capable of forming at least two hydrogen bonds with said phenol groups of the polyphenol X, which is preferably nonionic, polyoxyethylenated and/or polyoxypropylenated and/or (poly)glycerolated; c) at least one C2-C8, preferably C2-C5, monoalcohol, preferably ethanol or isopropanol; d) at least one acid chosen from phytic acid, acetic acid, at least one hydroxylated carboxylic acid chosen from C6 aromatic or C2-C10 non-aromatic monocarboxylic or polycarboxylic acids, bearing at least one free OH radical in the alpha or beta position of a carboxylic group that is free or engaged in a heterocycle; ferulic acid; and also mixtures thereof. The invention also relates to a method for making up and/or caring for the skin and/or the lips, in particular the lips, in which the abovementioned composition is applied.

The present invention relates to a composition for making up and/or caring for the skin and/or the lips, comprising at least one compound of polyphenol type, at least one compound Y capable of forming hydrogen bonds with the polyphenol and comprising polyoxyalkylenated and/or (poly)glycerolated units, at least one C2-C8 alcohol, and at least one particular hydroxylated carboxylic acid.

For several years, consumers have been increasingly demanding as regards the composition of their cosmetic products and are seeking in particular to use products comprising an ever greater content of natural ingredients or ingredients of natural origin, of ingredients which have a minimized environmental impact and/or ingredients which are compatible with numerous packagings.

The presence of polyphenols and in particular those of natural origin, is particularly desired for their beneficial properties for keratin materials. However, the compositions comprising such compounds do not give complete satisfaction in terms of stability over time, in particular when they contain significant contents of short (in particular C2-C8) alcohols. Specifically, this type of compound, in an alcoholic medium, more especially an ethanolic medium, is sensitive to hydrolysis reactions and transesterification reactions. These reactions are the cause of the appearance, over time, of gallic acid and esters thereof, such as for example ethyl gallate. However, gallic acid and esters thereof are known for their sensitizing potential as regards the skin and lips, and therefore the respective content thereof in the compositions must be and remain very limited over time.

The need thus remains to find novel polyphenol-based compositions for caring for and/or making up keratin materials which are stable over time, and which produce, after application to the skin and/or the lips, a homogeneous deposit and pleasant sensory properties such as the perception of a very thin film, a good wear property over the time of the effect expected by the composition, without the drawbacks mentioned above.

These objectives and others are achieved by the present invention which relates to a composition for making up and/or caring for the skin and/or the lips, preferably the lips, more particularly a makeup composition, comprising:
a) at least one polyphenol X comprising at least two different phenol groups;
b) at least one compound Y chosen from those capable of forming at least two hydrogen bonds with said phenol groups of the polyphenol X, which is preferably nonionic, polyoxyethylenated and/or polyoxypropylenated and/or (poly)glycerolated;
c) at least one C2-C8, preferably C2-C5, monoalcohol, preferably ethanol or iso-propanol;
d) at least one acid chosen from phytic acid, acetic acid, at least one hydroxylated carboxylic acid chosen from C6 aromatic or C2-C10 non-aromatic monocarboxylic or polycarboxylic acids, bearing at least one free OH radical in the alpha or beta position of a carboxylic group that is free or engaged in a heterocycle; from ferulic acid; and also mixtures thereof.

The invention also relates to a method for making up and/or caring for the skin and/or the lips, in particular the lips, in which the abovementioned composition is applied.

Other aspects and advantages of the present invention will emerge more clearly on reading the description and the examples that follow.

In the context of the present invention, it is indicated that the skin denotes the skin of the face (cheeks, eyelids, eye contour), of the body and of the hands.

The compositions for making up and/or caring for the skin and/or the lips according to the invention are cosmetic compositions. This implies that they advantageously comprise a physiologically acceptable medium. The term “physiologically acceptable” is intended to mean compatible with the skin and/or the lips, having a pleasant color, odor and feel and not causing any unacceptable discomfort (stinging or tautness) liable to discourage the consumer from using this composition.

For the purposes of the invention, the term “hydrogen bonding interaction” means an interaction involving a hydrogen atom of one of the two reagents and an electronegative heteroatom of the other reagent, such as oxygen, nitrogen, sulfur and fluorine. In the context of the invention, the hydrogen bond(s) is (are) formed between the hydroxyl (OH) function(s) of the reactive phenol group(s) of the polyphenol X and the hydroxyl group(s) of the compound Y capable of reacting by hydrogen bonding with the polyphenol X.

The present invention more particularly comprises the use of a coating agent obtained by hydrogen bonding interaction of at least one polyphenol X with at least one compound Y. More particularly, said coating agent is in the latent state in the composition according to the invention and only fully appears in situ, i.e. once the composition has been applied on the skin and/or the lips. In other words, in the composition according to the invention, before it is applied, the conditions (ingredients, contents) are such that they do not promote the interaction of the polyphenol(s) X and compound(s) Y with one another. Preferably, these compounds do not precipitate in the composition before it is applied.

The composition according to the invention is advantageously in a liquid form at room temperature.

The term “room temperature” means 25° C.

The term “atmospheric pressure” means 760 mmHg, i.e. 1.013×10⁵ pascals.

### Polyphenol X

The polyphenols X that can be used according to the present invention include in their structure at least two different phenol groups.

A “polyphenol” is understood to mean any compound having in its chemical structure at least two benzene compounds (i.e.: groups), in free or fused form, each benzene compound comprising at least one hydroxyl (OH) group, preferably at least 2 hydroxyl groups, or even 3 hydroxyl groups.

The term “different phenol groups” refers to phenol groups that are chemically different.

The polyphenols X that may be used according to the invention may be synthetic or natural. They may be in isolated form or contained in a mixture, notably contained in a plant extract. Polyphenols are phenols comprising at least two phenol groups that are differently substituted on the aromatic ring.

The two classes of polyphenols are flavonoids and non-flavonoids.

Examples of flavonoids that may be mentioned include chalcones such as phloretin, phloridzin, aspalathin or neohesperidin; flavanols such as catechin, fisetin, kaempferol, myricetin, quercetin, rutin, procyanidins, proanthocyanidins, pyroanthocyanidins, theaflavins or thearubigins (or thearubins); dihydroflavonols such as astilbin, dihydroquercetin (taxifolin) or silibinin; flavanones such as hesperidin, neohesperidin, hesperetin, naringenin or naringin; anthocyanins such as cyanidin, delphinidin, malvidin, peonidin or petunidin; catechin tannins such as tannic acid; isoflavonoids such as daidzein or genistein; neoflavanoids; lignans such as pyroresorcinol; and mixtures thereof.

Among the natural polyphenols X that may be used according to the invention, mention may also be made of lignins.

Examples of non-flavonoids that may be mentioned include curcuminoids such as curcumin or tetrahydrocurcumin; stilbenoids such as astringin, resveratrol or rhaponticin; aurones such as aureusidin; and mixtures thereof.

As polyphenols X that can be used according to the invention, mention may also be made of chlorogenic acid, verbascoside; coumarins substituted with phenols.

According to a particular embodiment of the invention, the polyphenol will be chosen from catechin tannins such as gallotannins chosen from tannic acid; ellagitannins such as epigallocatechin, epigallocatechin gallate, castalagin, vescalagin, vescalin, castalin, casuarictin, castanopsinins, excoecarianins, grandinin, gradinin, roburins, pterocarinin, acutissimin, tellimagrandins, sanguiin, potentillin, pedunculagin, geraniin, chebulagic acid, repandusinic acid, ascorgeraniin, stachyurin, casuarinin, casuariin, punicacortein, coriariin, cameliatannin, isodehydrodigalloyl, dehydrodigalloyl, hellinoyl, punicalagin and rhoipteleanins.

According to a particular embodiment of the invention, the polyphenol X is epigallocatechin, in particular a green tea extract having the INCI name Green Tea Extract, notably comprising at least 45% epigallocatechin relative to the total weight of said extract, for instance the commercial product sold under the name Dermofeel Phenon 90 M-C® sold by Evonik Nutrition & Care or the commercial product sold under the name Tea Polyphenols Green Tea Extract® by Tayo Green Power.

According to a particular embodiment of the invention, the polyphenol X is a procyanidin or a mixture of procyanidins, in particular an extract of maritime pine bark having the INCI name Pinus pinaster Bark/Bud Extract, notably comprising at least 65% by weight of procyanidins relative to the total weight of said extract, such as the commercial product sold under the name Pycnogenol® sold by Biolandes Arômes.

Tannic acid will be used more particularly as polyphenol X. This compound is notably sold under the name Brewtan F by Ajinomoto Omnichem NV.

Preferably, the content of polyphenol is at least 2% by weight, preferably between 2% and 30% by weight, and even more particularly from 3% to 25% by weight, relative to the total weight of said composition.

### Compounds Y

The compound(s) Y capable of forming at least two hydrogen bonds with said phenol groups of the polyphenol X are therefore polyoxyethylenated and/or poly-oxypropylenated and/or polyglycerolated hydrocarbon-based or silicone compounds.

According to a particular embodiment, the compound(s) Y, in the medium of the composition, do not comprise any anionic group in their chemical structure, and in particular are nonionic.

Preferably, the compound(s) Y is/are chosen from compounds having a molar mass of greater than 200 g/mol, more particularly greater than 350 g/mol.

They are more particularly chosen from polyoxyethylenated and/or poly-oxypropylenated and/or polyglycerolated linear, branched or crosslinked silicone compounds, polyoxyethylenated and/or polyoxypropylenated alcohols, polyoxyethylenated and/or polyoxypropylenated alkylglycols or glycerol, polyethylene glycols, Poloxamers, polyoxyethylenated and/or polyoxypropylenated and/or polyglycerolated esters, polyoxyethylenated esters of sorbitol or sorbitan and polysorbates, polyoxyethylenated sugar ester or ether derivatives, polyoxyethylenated (poly)amines, and also mixtures thereof.

Polyoxyethylenated and/or Polyoxypropylenated and/or Polyglycerolated Silicone Compounds

Among suitable compounds can be cited polyoxyethylenated and/or polyoxypropylenated and/or polyglycerolated linear, branched or crosslinked nonionic polydimethylsiloxanes comprising from 2 to 50 oxyethylene units and/or comprising from 2 to 50 oxypropylene units and/or comprising from 2 to 20 glycerol units, optionally comprising an alkyl group comprising from 6 to 22 carbon atoms; polyoxyethylenated nonionic polydimethylsiloxanes with ester function(s), comprising from 2 to 50 oxyethylene units; and also the mixtures thereof.

Preferably, the compound(s) Y is/are chosen from the following compounds, denoted by their INCI name: PEG-10 Dimethicone, PEG-12 Dimethicone, PEG-9 Polydimethylsiloxyethyl Dimethicone, Lauryl PEG-9 Polydimethylsiloxyethyl Dimethicone, Dimethicone/PEG-10/15 Crosspolymer, PEG/PPG-17/18 Dimethicone, PEG/PPG-18/18 Dimethicone, PEG/PPG-22/24 Dimethicone, Bis-PEG-12 Dimethicone Candelillate, Bis-PEG-12 Dimethicone Beeswax, Dimethicone/Polyglycerin-3 Crosspolymer and also the mixtures thereof.

Polyoxyethylenated and/or Polyoxypropylenated Alcohols

Among the compounds of this type, mention may be made of the following, alone or as mixtures:
polyoxyethylenated fatty alcohols of R(O—CH₂—CH₂)ₒ—OH type, R representing a C₈-C₃₀ alkyl radical, o representing an average integer ranging from 2 to 50, notably chosen from Ceteth-2, Ceteth-10, Ceteth-20, Ceteth-25, Ceteth-40, Isoceteth-20, Laureth-2, Laureth-3, Laureth-4, Laureth-12, Laureth-23, Oleth-2, Oleth-5, Oleth-10, Oleth-20, Oleth-25, Deceth-3, Deceth-5, Beheneth-10, Steareth-2, Steareth-10, Steareth-20, Steareth-21, Steareth-100, Ceteareth-12, Ceteareth-15, Ceteareth-20, Ceteareth-25, Ceteareth-30, Ceteareth-33, Coceth-7, Trideceth-12 and the mixtures thereof.

Polyoxyethylenated and/or oxypropylenated alcohols of the following type: R—(O—C(CH₃)H—CH₂)ₒ—(O—CH₂—CH₂)ₚ—OH; R representing a C₄-C₃₀ alkyl radical, o and p, independently of one another, representing an average integer ranging from 1 to 50. Preferably, the compound Y is chosen from the compounds whose INCI name is as follows: PPG-26-Buteth-26, PPG-12-Buteth-16, PPG-5-Ceteth-20, PPG-4-Ceteth-20, PPG-6-Decyltetradeceth-30, and the mixtures thereof.

Polyoxyethylenated and/or Polyoxypropylenated Alkylglycols or Glycerol

Mention may notably be made of the following, alone or as mixtures:

Polyoxyethylenated glycerols, in particular glycerol oxyethylenated with 26 OE (Glycereth-26).

Polyoxyalkylenated alkanediols such as PEG-8 Caprylyl Glycol.

Compounds of the type: R—(O—CH₂—CH₂)ₒ—O—CH₂—CH(R′)OH, in particular Ceteareth-60 Myristyl Glycol.

Polyoxyethylenated and/or polyoxypropylenated alkyl glycol ethers such as PPG-1-PEG-9 Lauryl Glycol Ether.

Compounds of the type: H(O—CR—CH₂)ₒ—(CH₂—CH₂—O)ₚ—(CH₂—C(R)H—O)ᵣH, in particular PEG-45/Dodecyl Glycol Copolymer.

### Polyethylene Glycols

As regards these compounds, the latter may be chosen, alone or as mixtures, from the polyethylene glycols of H(O—CH₂—CH₂)ₙ—OH type, in particular chosen from PEG-6, PEG-8, PEG-14M, PEG-20, PEG-45M, PEG-90, PEG-90M, PEG-150, PEG-180, PEG-220, and mixtures thereof.

### Poloxamers

Suitable for the implementation of the invention are Poloxamers (INCI name) which correspond in particular to the following formula: HO—(CH₂—CH₂—O)ₙ—(CHCH₃—CH₂—O) ₒ—(CH₂—CH₂—O)ₚ—H, in particular chosen from Poloxamer 124, Poloxamer 184, Poloxamer 338, and also the mixtures thereof.

Polyoxyethylenated and/or Polyoxypropylenated and/or Polyglycerolated Esters

Suitable for example are:

Acid esters of polyethylene glycol of the type:
R—CO—(O—CH₂—CH₂)ₙ—OH or
R—CO—(O—CH₂—CH₂)_{q}—O—CO—R or
R—CO—(O—CH₂—CH₂)_{q}—O—R or
R—O—(CH(CH₃)—(CH₂))ₚ—(O—CH₂—CH₂)_{q}—O—CO—R
   in which R, which are identical or different, represent saturated or unsaturated C2-C20 hydrocarbon-based groups; n, p, q, which are average integers and are identical or different, vary from 2 to 150.

They may in particular be chosen from PEG-6 Isostearate, PEG-6 Stearate, PEG-8 Stearate, PEG-8 Isostearate, PEG-20 Stearate, PEG-30 Stearate, PEG-32 Stearate, PEG-40 Stearate, PEG-75 Stearate, PEG-100 Stearate, PEG-8 Distearate, PEG-150 Distearate, Mereth-3 Myristate, PEG-4 Olivate, Propylene Glycol Ceteth-3 Acetate, PEG-30 Dipolyhydroxystearate, and also the mixtures thereof.

Esters of glycerol and of optionally hydroxylated C₆-C₄₀, more particularly C₈-C₃₀, carboxylic acid(s) or carboxylic acid polymer(s), or derivatives of plant oils, said esters being polyoxyethylenated, comprising from 2 to 200 oxyethylene units, more preferentially from 2 to 100 oxyethylene units, even more particularly between 2 and 80 oxyethylene units. These compounds are more particularly in the form of monoglycerides, diglycerides or triglycerides, alone or as mixtures. As examples of such esters, mention may be made of PEG-6 Caprylic/Capric Glycerides, PEG-60 Almond Glycerides, PEG-10 Olive Glycerides, PEG-45 Palm Kernel Glycerides, PEG-7 Glyceryl Cocoate, PEG-30 Glyceryl Cocoate, PEG-40 Hydrogenated Castor Oil, PEG-60 Hydrogenated Castor Oil, PEG-30 Glyceryl Stearate, PEG-200 Glyceryl Stearate, PEG-20 Glyceryl Triisostearate, PEG-70 Mango Glycerides, Hydrogenated Palm/Palm Kernel Oil PEG-6 Esters, PEG-200 Hydrogenated Glyceryl Palmate, PEG-7 Glyceryl Cocoate, the mixture of polyoxyethylenated (200 OE) palm glycerides and of polyoxyethylenated (7 OE) coconut glycerides, and also the mixtures thereof.

Esters of polyglycerol comprising 2 to 20 glycerol units and of saturated or unsaturated carboxylic acid(s) or carboxylic acid polymer(s) comprising from 6 to 40 carbon atoms, preferably 8 to 30 carbon atoms, or esters of polyglycerol comprising 2 to 20 glycerol units and derivatives of plant oils, and also the mixtures thereof. Preferably, the compounds Y are chosen from polyglycerolated esters comprising 2 to 20 glycerol units and of saturated or unsaturated carboxylic acid(s), comprising 6 to 40 carbon atoms, preferably 8 to 30 carbon atoms, or else derivatives of plant oils, and the mixtures thereof. The carboxylic acids may further comprise 1 to 3 carboxylic groups, and preferably they are monocarboxylic acids. The polyglycerolated compounds are more particularly monoesters, diesters or triesters. As examples, mention may be made of the following compounds, denoted by their INCI name: Polyglyceryl-2 Stearate, Polyglyceryl-2 Isostearate, Polyglyceryl-2 Diisostearate, Polyglyceryl-3 Diisostearate, Polyglyceryl-3 Dicitrate/Stearate, Polyglyceryl-4 Diisostearate, Polyglyceryl-4 Caprate, Polyglyceryl-4 Laurate, Polyglyceryl-5 Laurate, Poylglyceryl-5 Oleate, Polyglyceryl-6 Caprylate, Polyglyceryl-6 Dicaprate, Polyglyceryl-6 Distearate, Polyglyceryl-6 Caprylate/Caprate, Polyglyceryl-6 Dioleate, Polyglyceryl-6 Trilaurate, Polyglyceryl-10 Laurate, Polyglyceryl-10 Dioleate, polyglycerolated apricot kernel oil esters comprising from 3 to 10 glycerol units, and also the mixtures thereof.

Polyoxyethylenated butters, in particular polyoxyethylenated shea butter.

Polyoxyethylenated waxes, notably chosen from polyoxyethylenated ester waxes such as polyoxyethylenated (120 OE) jojoba wax (INCI name: Jojoba Wax PEG-120 Esters), PEG-8 Beeswax, PEG-60 Lanolin, PEG-75 Lanolin, PPG-12-PEG-50 Lanolin, and the mixtures thereof.

Polyoxyethylenated dihydrocholesteryl esters, in particular Dihydrocholeth-30.

PEG-55 Propylene Glycol Oleate.

Polyoxyethylenated pentaerythritol esters, in particular chosen from PEG-150 Pentaerythrityl Tetrastearate.

Polyoxyethylenated glycerolated esters such as Glycereth-25 PCA Isostearate.

Polyoxyethylenated lanolins such as Laneth-15.

Mixtures thereof.

### Polyoxyethylenated Esters of Sorbitol or Sorbitan and Polysorbate

These compounds are more particularly chosen from the esters of sorbitol or sorbitan and of saturated or unsaturated C₆-C₄₀, advantageously C₈-C₃₀, carboxylic acid(s) and comprising 2 to 50 oxyethylene units.

Among the Polysorbates (INCI name), mention may very particularly be made of the compounds having the following INCI names: Polysorbate-20, Polysorbate-21, Polysorbate-60, Polysorbate-61, Polysorbate-80, Polysorbate-85, and also the mixtures thereof. As regards the esters, mention may be made of PEG-40 Sorbitan Peroleate.

### Polyoxyethylenated Sugar Ester or Ether Derivatives

The sugar derivatives are more particularly glucose derivatives such as for example polyoxyethylenated alkylglucoses, for instance the compounds defined by the following INCI names: Methyl-Gluceth-10, Methyl-Gluceth-20. Also suitable are the polyoxyethylenated sugar esters such as for example the compounds having the following INCI names: PEG-120 Methyl Glucose Diolcate, PEG-20 Methyl Glucose Sesquistearate, and also mixtures thereof.

### Polyoxyethylenated (Poly)Amines

Suitable for the implementation of the invention are polyoxyethylenated alkylamines, more particularly of formula R—N[(CH₂—CH₂—O)H]_{q}[(CH₂—CH₂—O)ᵣH] with R representing a saturated or unsaturated C8-C30 hydrocarbon-based group, and q or r, which are identical or different, represent an average integer ranging from 1 to 50 and in particular PEG-2-Oleamine. Polyoxyethylenated polyamines such as for example PEG-15 Cocopolyamine may also be suitable.

According to a particularly preferred embodiment of the invention, the compound(s) Y are chosen from:
polyoxyethylenated fatty alcohols of R(O—CH₂—CH₂)ₒ—OH type, R representing a C₈-C₃₀ alkyl radical, o representing an average integer ranging from 2 to 50;
polyoxyethylenated and/or oxypropylenated alcohols of the following type: R—(O—C(CH₃)H—CH₂)ₒ—(O—CH₂—CH₂)ₚ—OH; R representing a C₄-C₃₀ alkyl radical, o and p, independently of one another, representing an average integer ranging from 1 to 50;
esters of polyglycerol comprising 2 to 20 glycerol units and of saturated or unsaturated carboxylic acid(s) or carboxylic acid polymer(s) comprising from 6 to 40 carbon atoms; or derivatives of plant oils;
esters of glycerol and of saturated or unsaturated C₆-C₄₀, more particularly C₈-C₃₀, carboxylic acid(s) or carboxylic acid polymer(s) or derivatives of plant oils, said esters being (poly)oxyethylenated, comprising from 1 to 200 oxyethylene units, more particularly 1 to 100, or even 1 to 80 oxyethylene units;
polysorbates;
mixtures thereof.

According to a very advantageous variant of the invention, the compound(s) Y are chosen from esters of polyglycerol comprising 2 to 20 glycerol units and of saturated or unsaturated carboxylic acid(s) or carboxylic acid polymer(s) comprising from 6 to 40 carbon atoms. Even more preferably, they are chosen from the following compounds, denoted by their INCI name: Polyglyceryl-2 Stearate, Polyglyceryl-2 Isostearate, Polyglyceryl-2 Diisostearate, Polyglyceryl-3 Diisostearate, Polyglyceryl-3 Dicitrate/Stearate, Polyglyceryl-4 Diisostearate, Polyglyceryl-4 Caprate, Polyglyceryl-4 Laurate, Polyglyceryl-5 Laurate, Poylglyceryl-5 Oleate, Polyglyceryl-6 Caprylate, Polyglyceryl-6 Dicaprate, Polyglyceryl-6 Distearate, Polyglyceryl-6 Caprylate/Caprate, Polyglyceryl-10 Laurate, Polyglyceryl-10 Dioleate, and also the mixtures thereof.

In accordance with one particular embodiment of the invention, the content of compound(s) Y represents at least 1% by weight, more particularly at least 2% by weight, preferably from 2% to 40% by weight and even more preferentially between 3% and 35% by weight, relative to the total weight of said composition.

More particularly, the ratio of the mass of polyphenol(s) X, expressed as active material, to the mass of the compounds Y, expressed as active material, varies between 0.25 and 3, preferably between 0.5 and 2.

### C₂-C₈ Monoalcohol

The composition according to the invention comprises at least one C₂-C₈, more particularly C₂-C₅ monoalcohol. As examples, mention may be made of ethanol, isopropanol, butanol, and preferably ethanol and isopropanol, and even more preferentially ethanol.

Advantageously, the content of C₂-C₈ monoalcohol is such that the compounds X and Y do not react together in the composition, before it is applied. Said monoalcohol may thus be considered to be a hydrogen bond inhibitor. The term “hydrogen bond inhibitor” is understood to mean any compound which is capable of preventing the hydrogen bonding interaction between the polyphenol X and the compound Y and/or which is capable of dissociating the complex formed by said interaction by breaking the hydrogen bonds.

More particularly, the content of C₂-C₈, preferably C₂-C₅ monoalcohol and more particularly ethanol, is greater than 25% by weight, preferably between 25% and 98% by weight, more particularly between 30% and 85% by weight and even more particularly between 35% and 80% by weight, relative to the total weight of the composition.

### Acid

As indicated above, the composition comprises at least one acid chosen from phytic acid, acetic acid, at least a hydroxylated carboxylic acid chosen from non-aromatic C2-C10 or aromatic C6 mono- or poly-carboxylic acids, carrying at least one free OH radical in the alpha or beta position of a free carboxylic group or engaged in a hetcrocycle; ferulic acid, as well as mixtures thereof.

### Phytic Acid, Acetic Acid

According to ta first embodiment, the composition according to the invention further comprises at least one acid chosen from phytic acid, acetic acid, and also mixtures thereof.

Preferably, the composition comprises at least phytic acid. More particularly, it is recalled that this acid, of natural origin, corresponds to the hexaphosphoric ester of inositol (C6 cyclic saturated alcohol, each carbon atom comprising a hydroxyl group). This product is notably sold under the name Tsuno Rice Fine Chemicals Phytic Acid (IP6).

Preferably, said acid(s) is (are) in an acid form in the composition.

More particularly, the content of phytic and/or acetic acid, expressed as active material, varies from 0.1% to 6%, more particularly from 0.2% to 5% by weight, relative to the total weight of the composition.

### Hydroxylated Carboxylic Acid

According to a second embodiment, the composition according to the invention further comprises at least one hydroxylated carboxylic acid chosen from C6 aromatic or C2-C10 non-aromatic monocarboxylic or polycarboxylic acids, bearing at least one free OH radical in the alpha or beta position of a carboxylic acid group that is free or engaged in a heterocycle; from ferulic acid; and also mixtures thereof.

More particularly, the hydroxylated carboxylic acid comprises a carboxylic group (also referred to interchangeably as a carboxylic acid group) engaged in a heterocycle or comprises 1 to 4 free carboxylic groups.

More particularly, the number of free hydroxyl radicals varies from 1 to 4.

Also suitable for the implementation of the invention are hydroxylated carboxylic acids, which may also comprise an ester function. This ester function is more particularly obtained by reaction of a free hydroxyl radical with a saturated or unsaturated carboxylic acid comprising 2 to 20 carbon atoms.

According to a first variant, the hydroxylated carboxylic acid is chosen from C₂-C₁₀ non-aromatic monocarboxylic or polycarboxylic acids bearing at least one OH radical in the alpha position of a carboxylic acid group that is free or engaged in a heterocycle; the hydroxylated carboxylic acid comprising a carboxylic acid group engaged in a hetcrocycle or comprising 1 to 4 free carboxylic acid groups; the number of free hydroxyl radicals varying from 1 to 4; it being possible for said hydroxylated carboxylic acid to optionally comprise an ester function.

Preferably, the hydroxylated acid according to this variant is chosen from alphahydroxy acids such as in particular erythorbic acid, ascorbic acid, citric acid, lactic acid, and also isomers thereof; glycolic acid, and also mixtures thereof. As an example of a hydroxylated carboxylic acid also comprising an ester function, mention may be made of the compounds obtained from ascorbic or erythorbic acid, bearing an ester function on one of the free hydroxyl radicals of the dihydroxyethyl group, and very particularly ascorbyl palmitate.

According to another variant, the hydroxylated acid is chosen from C6 aromatic monocarboxylic or polycarboxylic acids bearing an OH radical in the beta position of a free carboxylic acid group, such as more particularly salicylic acid.

Preferably, the composition comprises at least one alpha-hydroxy acid, preferably not comprising an ester function, or a mixture of these.

More particularly, the content of hydroxylated carboxylic acid varies from 0.1% to 6%, more particularly from 0.2% to 5% by weight, relative to the total weight of the composition.

### Water and Water-Soluble Solvents

The composition can optionally comprise water. This may be a demineralized water or alternatively a floral water such as cornflower water and/or a mineral water such as Vittel water, Lucas water or La Roche Posay water and/or a spring water.

More particularly, the water content is less than or equal to 20% by weight, preferably less than or equal to 15% by weight of water, is less than or equal to 10% by weight, relative to the total weight of the composition.

In accordance with one particularly advantageous embodiment of the invention, the water content of the composition is between 0 and 5% by weight, limits included, relative to the total weight of said composition.

In accordance with a preferred embodiment of the invention, the water content is less than 5% by weight, more particularly less than 2% by weight, and more particularly still less than 1% by weight, relative to the weight of the composition. According to one particular embodiment, the composition is anhydrous. It should be noted that in such a case, the water is not added deliberately to the composition, but may be present in small amounts, or even in trace amounts, in the various products used.

If the composition comprises water, then the pH of the aqueous phase is advantageously less than 8.0, more preferentially less than 7.0, and more particularly varies from 2 to 6.

The composition may optionally comprise other water-soluble or water-miscible ingredients or solvents (miscibility with water of greater than 50% by weight at 25° C.), for instance polyols having from 3 to 8 carbon atoms such as propylene glycol, 1,3-butylene glycol, caprylyl glycol, pentylene glycol, glycerol, and dipropylene glycol; C₃-C₄ ketones and C₂-C₄ aldehydes.

### Colorant

The composition according to the invention may optionally comprise at least one colorant, which is synthetic, natural or of natural origin.

More particularly, the content of colorant(s) in the composition is advantageously at least 0.01% by weight, more particularly between 0.05% and 30% by weight, in particular between 0.1% and 25% by weight, relative to the total weight of the composition.

The colorant(s) may be chosen from coated or uncoated pigments, water-soluble dyes, liposoluble dyes, and mixtures thereof.

### Pigments

The term “pigments” means white or colored, mineral or organic particles, which are insoluble in the medium of the composition, and which are intended to color and/or opacify the resulting composition and/or deposit.

According to a first particular embodiment, the pigments used are chosen from mineral pigments.

The term “mineral pigment” means any pigment that satisfies the definition in Ullmann's encyclopedia in the chapter on inorganic pigments. Mention may be made, among the mineral pigments of use in the present invention, of zirconium or cerium oxides, and also zinc, iron (black, yellow or red) or chromium oxides, manganese violet, ultramarine blue, chromium hydrate and ferric blue, titanium dioxide, or metal powders, such as aluminum powder and copper powder. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

The size of the pigment of use in the context of the present invention is generally greater than 100 nm and can range up to 10 um, preferably from 200 nm to 5 um and more preferentially from 300 nm to 1 μm.

According to a particular form of the invention, the pigments have a size characterized by a D[50] of greater than 100 nm and possibly ranging up to 10 μm, preferably from 200 nm to 5 μm and more preferentially from 300 nm to 1 μm.

The sizes are measured by static light scattering using a commercial MasterSizer 3000® particle size analyzer from Malvern, which makes it possible to determine the particle size distribution of all of the particles over a wide range which may extend from 0.01 μm to 1000 μm. The data are processed on the basis of the standard Mie scattering theory. This theory is the most suitable for size distributions ranging from submicron to multimicron; it allows an “effective” particle diameter to be determined. This theory is described in particular in the publication by Van de Hulst, H.C., Light Scattering by Small Particles, Chapters 9 and 10, Wiley, New York, 1957.

D[50] represents the maximum size exhibited by 50% by volume of the particles.

In the context of the present invention, the mineral pigments are more particularly iron oxide and/or titanium dioxide. Examples that may be mentioned more particularly include titanium dioxide and iron oxide coated with aluminum stearoyl glutamate, sold, for example, under the reference NAI® by the company Miyoshi Kasei.

As mineral pigments that may be used in the invention, mention may also be made of nacres.

The term “nacres” should be understood as meaning colored particles of any form, which may or may not be iridescent, notably produced by certain molluscs in their shell, or alternatively synthesized, and which have a color effect via optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles, at the surface of which are superposed at least two successive layers of metal oxides and/or of organic colorants.

Examples of nacres that may also be mentioned include natural mica covered with titanium oxide, with iron oxide, with natural pigment or with bismuth oxychloride.

The nacres can more particularly have a yellow, pink, red, bronze, orangey, brown, gold and/or coppery color or tint.

Among the pigments that can be used according to the invention, mention may also be made of those having an optical effect different from a simple conventional coloring effect, i.e. a unified and stabilized effect such as produced by conventional colorants, for instance monochromatic pigments. For the purposes of the invention, the term “stabilized” means lacking the effect of variability of the color with the angle of observation or in response to a temperature change.

For example, this material may be chosen from particles with a metallic tint, goniochromatic coloring agents, diffractive pigments, thermochromic agents, optical brighteners, and also fibers, notably interference fibers. Needless to say, these various materials may be combined in order simultaneously to afford two effects, or even a novel effect in accordance with the invention.

According to a particular embodiment, composition (A), (B), (C) and/or (D) according to the invention comprises at least one uncoated pigment.

According to another particular embodiment, composition (A), (B), (C) and/or (D) according to the invention comprises at least one pigment coated with at least one lipophilic or hydrophobic compound.

This type of pigment is particularly advantageous. Insofar as they are treated with a hydrophobic compound, they show predominant affinity for an oily phase, which can then convey them.

The coating can also comprise at least one additional nonlipophilic compound.

For the purposes of the invention, the “coating” of a pigment according to the invention generally denotes the total or partial surface treatment of the pigment with a surface agent, absorbed on, adsorbed on or grafted to said pigment.

The surface-treated pigments can be prepared according to surface treatment techniques of chemical, electronic, mechanochemical or mechanical nature which are well known to a person skilled in the art. Commercial products can also be used.

The surface agent can be absorbed on, adsorbed on or grafted to the pigments by solvent evaporation, chemical reaction and creation of a covalent bond.

According to one variant, the surface treatment consists in coating the pigments.

The coating may represent from 0.1% to 20% by weight and in particular from 0.5% to 5% by weight relative to the total weight of the coated pigment.

The coating may be produced, for example, by adsorption of a liquid surface agent onto the surface of the solid particles by simple mixing with stirring of the particles and of said surface agent, optionally with heating, prior to the incorporation of the particles into the other ingredients of the makeup or care composition.

The coating may be produced, for example, by chemical reaction of a surface agent with the surface of the solid pigment particles and creation of a covalent bond between the surface agent and the particles. This method is notably described in patent U.S. Pat. No. 4,578,266.

The chemical surface treatment may consist in diluting the surface agent in a volatile solvent, dispersing the pigments in this mixture and then slowly evaporating off the volatile solvent, so that the surface agent is deposited on the surface of the pigments.

When the pigment comprises a lipophilic or hydrophobic coating, the latter is preferably present in the fatty phase of the composition according to the invention.

According to a particular embodiment of the invention, the pigments may be coated according to the invention with at least one compound chosen from silicone surface agents; fluoro surface agents; fluorosilicone surface agents; metal soaps; N-acylamino acids or salts thereof; lecithin and derivatives thereof; isopropyl triisostearyl titanate; isostearyl sebacate; natural plant or animal waxes; polar synthetic waxes; fatty esters; phospholipids; and mixtures thereof.

According to a particular embodiment of the invention, the pigments may be coated with a hydrophilic compound.

According to another particular embodiment, the colorant is an organic pigment, which is synthetic, natural or of natural origin.

The term “organic pigment” refers to any pigment that satisfies the definition in Ullmann's encyclopedia in the chapter on organic pigments. The organic pigment can in particular be chosen from nitroso, nitro, azo, xanthene, quinoline, anthraquinone, phthalocyanine, of metal complex type, isoindolinone, isoindoline, quinacridone, perinone, perylene, diketopyrrolopyrrole, thioindigo, dioxazine, triphenylmethane or quinophthalone compounds.

The organic pigment(s) may be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as described in patent FR 2 679 771.

The pigments may also be in the form of composite pigments as described in patent EP 1 184 426. These composite pigments may notably be composed of particles including a mineral core at least partially covered with an organic pigment and at least one binder for fixing the organic pigments to the core.

The pigment can also be a lake. The term “lake” is understood to mean insolubilized dyes adsorbed on insoluble particles, the assembly thus obtained remaining insoluble during use.

The inorganic substrates onto which the dyes are adsorbed are, for example, alumina, silica, calcium sodium borosilicate or calcium aluminum borosilicate and aluminum.

Among the organic dyes, mention may be made of cochincal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

Mention may be made, by way of an example of a lake, of the product known under the name D&C Red 7 (CI 15 850:1).

The pigment(s) are preferably present at contents of at least 0.01% by weight, more particularly of at least 1% by weight, and even more particularly of at least 2% by weight, relative to the weight of the composition in question. More particularly, the content of colorant is less than 30% by weight, and more particularly between 0.05% and 30% by weight, and even better still from 0.1% to 25% by weight, relative to the total weight of the composition.

### Water-Soluble or Liposoluble Dyes

According to another particular embodiment of the invention, the colorant is a water-soluble dye or a liposoluble dye.

For the purposes of the invention, a “water-soluble colorant” is understood to mean any natural or synthetic, generally organic compound, which is soluble in an aqueous phase or water-miscible solvents and which is capable of imparting color.

For the purposes of the invention, a “liposoluble colorant” is understood to mean any natural or synthetic, generally organic compound, which is soluble in an oily phase or in solvents that are miscible with the oily phase, and which is capable of imparting color.

Mention may in particular be made, as water-soluble dyes which are suitable for the invention, of synthetic or natural water-soluble dyes, such as, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5 or FDC Blue 1.

Among the natural water-soluble dyes, mention may be made of anthocyanins.

As liposoluble dyes that are suitable for use in the invention, mention may notably be made, for instance, of the liposoluble dyes DC Red 17, DC Red 21, DC Red 27, DC Green 6, DC Yellow 11, DC Violet 2, DC Orange 5, Sudan red and Sudan brown.

As illustrations of natural liposoluble dyes, mention may be made particularly of carotenes, for instance β-carotene, α-carotene and lycopene; quinoline yellow; xanthophylls such as astaxanthin, antheraxanthin, citranaxanthin, cryptoxanthin, canthaxanthin, diatoxanthin, flavoxanthin, fucoxanthin, lutein, rhodoxanthin, rubixanthin, siphonaxanthin, violaxanthin, zeaxanthin; annatto; curcumin; quinizarin (Ceres Green BB, D&C Green No. 6, CI 61565, 1,4-di-p-toluidinoanthraquinone, Green No. 202, quinazine green SS) and chlorophylls.

The water-soluble or liposoluble dye(s) are preferably present in the composition in contents of less than 4% by weight, or even less than 2% by weight, more preferentially ranging from 0.01% to 2% by weight and even better still from 0.02% to 1.5% by weight, relative to the total weight of the composition.

### Oily Phase

According to another particular embodiment of the invention, the composition according to the invention, which is preferably liquid, may comprise an oily phase.

The term “oily phase” refers to a phase which is liquid at room temperature and at atmospheric pressure, comprising at least one fatty substance, other than the compounds Y, such as oils, waxes or pasty substances and also optionally all the organic solvents and ingredients that are soluble or miscible in said phase.

### Oils

The oil(s) may be chosen from volatile or nonvolatile, polar or nonpolar, hydrocarbon-based oils, silicon oils, and mixtures thereof.

The term “oil” is understood to mean a fatty substance which is liquid at room temperature (25° C.) and atmospheric pressure (760 mmHg, i.e. 1.013×10⁵ Pa). The oil may be volatile or non-volatile.

Within the meaning of the present invention, the term “silicone oil” is understood to mean an oil comprising at least one silicon atom and in particular at least one Si—O group, and more particularly an organopolysiloxane.

The term “hydrocarbon-based oil” is understood to mean an oil containing mainly carbon and hydrogen atoms and possibly one or more functions chosen from hydroxyl, ester, ether or carboxyl functions. These oils are thus different from silicone oils.

For the purposes of the invention, the term “volatile oil” refers to any oil that is capable of evaporating on contact with the skin in less than one hour, at room temperature and atmospheric pressure. The volatile oil is a volatile cosmetic compound, which is liquid at room temperature, notably having a non-zero vapor pressure, at room temperature and atmospheric pressure, notably having a vapor pressure ranging from 2.66 Pa to 40 000 Pa, in particular ranging from 2.66 Pa to 13 000 Pa and more particularly ranging from 2.66 Pa to 1300 Pa.

The term “nonvolatile oil” refers to an oil that remains on the skin at room temperature and atmospheric pressure for at least several hours, and that notably has a vapor pressure of less than 2.66 Pa, preferably less than 0.13 Pa. By way of example, the vapor pressure may be measured via the static method or via the effusion method by isothermal thermogravimetry, depending on the vapor pressure (standard OCDE 104).

### Volatile Hydrocarbon-Based Oils

Among the hydrocarbon-based volatile oils, mention may more particularly be made of those chosen from hydrocarbon-based oils of hydrocarbon type (thus nonpolar hydrocarbon-based oils, consisting solely of carbon and hydrogen) and also of ester type. In particular, they may be chosen from volatile hydrocarbon-based oils containing from 8 to 16 carbon atoms, and mixtures thereof, and notably:
branched C₈-C₁₆ alkanes, such as isoalkanes (also known as isoparaffins), isododecane, isodecane, isohexadecane and mixtures thereof, and, for example, the oils sold under the trade names Isopar or Permethyl,
linear alkanes, for example C₁₁-C₁₅ alkanes, alone or as mixtures,
branched C₈-C₁₆ esters, for example isohexyl neopentanoate, and
mixtures thereof.

Other volatile hydrocarbon oils, such as petroleum distillates, in particular those sold under the name Shell Solt by Shell, can also be used; volatile linear alkanes, such as those described in the patent application DE10 2008 012 457 from Cognis.

### Volatile Silicone Oils

Among the volatile silicon oils, mention may be made, inter alia, of linear, branched or cyclic silicone oils such as polydimethylsiloxanes (PDMS) having from 3 to 7 silicon atoms, preferably linear or branched polydimethylsiloxanes having from 3 to 7 silicon atoms; and also the mixtures thereof.

Examples of such oils that may be mentioned include octyl trimethicone, hexyl trimethicone, methyl trimethicone, decamethylcyclopentasiloxane, octamethylcyclotetrasiloxane, dodecamethylcyclohexasiloxane, decamethyltetrasiloxane, polydimethylsiloxanes such as those sold under the reference DC 200 (1.5 cSt), or DC 200 (3 cSt) by Dow Corning or KF 96 A from Shin-Etsu; alone or as mixtures.

### Polar Nonvolatile Hydrocarbon-Based Oils

A “polar hydrocarbon-based oil” denotes an oil containing mainly hydrogen and carbon atoms and also comprising at least one oxygen atom. More particularly, such an oil comprises one or more functions chosen from hydroxyl, ester, ether or carboxyl functions, and preferably hydroxyl, ester or ether functions.

As examples of nonvolatile hydrocarbon-based oils that can be used in the invention, mention may be made of:
triglycerides constituted of fatty acid esters of glycerol, in particular the fatty acids of which may have chain lengths ranging from C4 to C36, and notably from C18 to C36, these oils possibly being linear or branched, and saturated or unsaturated; these oils may notably be heptanoic or octanoic triglycerides, wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil or musk rose oil; or alternatively caprylic/capric acid triglycerides, for instance those sold by Stearinerie Dubois or those sold under the names Miglyol 810®, 812® and 818® by Dynamit Nobel;
lincar aliphatic hydrocarbon-based esters of formula RCOOR′ in which RCOO represents a carboxylic acid residue including from 2 to 40 carbon atoms, and R′ represents a hydrocarbon-based chain containing from 1 to 40 carbon atoms, such as cetostearyl octanoate, isopropyl alcohol esters, such as isopropyl myristate or isopropyl palmitate, cthyl palmitate, 2-ethylhexyl palmitate, isopropyl stearate or isostearate, isostearyl isostearate, octyl stearate, heptanoates, and notably isostearyl heptanoate, alcohol or polyalcohol octanoates, decanoates or ricinoleates, for instance propylene glycol dioctanoate, cetyl octanoate, tridecyl octanoate, 2-ethylhexyl palmitate, alkyl benzoate, hexyl laurate, neopentanoic acid esters, for instance isodecyl neopentanoate, isotridecyl neopentanoate, isostearyl neopentanoate and 2-octyldodecyl neopentanoate, isononanoic acid esters, for instance isononyl isononanoate, isotridecyl isononanoate and octyl isononanoate, oleyl erucate, isocetyl stearate and isostearyl behenate;
polyesters obtained by condensation of unsaturated fatty acid dimer and/or trimer and of diol, such as those described in patent application FR 0 853 634, such as in particular of dilinoleic acid and of 1,4-butanediol. Mention may notably be made in this respect of the polymer sold by Biosynthis under the name Viscoplast 14436H® (INCI name: Dilinoleic Acid/Butanediol Copolymer), or else copolymers of polyols and of diacid dimers, and esters thereof, such as Hailuscent ISDA®;
linear fatty acid esters and polyesters containing a total carbon number ranging from 35 to 80, for instance pentaerythrityl tetrapelargonate or pentaerythrityl tetraisostearate;
aromatic esters and polyesters such as tridecyl trimellitate, C12-C15 alcohol benzoate, the 2-phenylethyl ester of benzoic acid, and butyloctyl salicylate;
esters and polyesters of diol dimer and of monocarboxylic or dicarboxylic acid, such as esters of diol dimer and of fatty acid and esters of diol dimer and of dicarboxylic acid dimer, for example of INCI name: Dimer Dilinoleyl Dimer Dilinoleate, such as Lusplan DD-DA5® and Lusplan DD-DA7® sold by Nippon Fine Chemical and described in application US 2004-175 338, the content of which is incorporated into the present application by reference;
fatty alcohols containing from 12 to 26 carbon atoms, for instance octyldodecanol, 2-butyloctanol, 2-hexyldecanol, 2-undecylpentadecanol or oleyl alcohol;
synthetic ethers containing from 10 to 40 carbon atoms, such as dicaprylyl ether;
dialkyl carbonates, the two alkyl chains possibly being identical or different, such as the dicaprylyl carbonate sold under the name Cetiol CC® by Cognis;
and mixtures thereof.

### Nonpolar Nonvolatile Hydrocarbon-Based Oils

The oil may also be chosen from linear or branched, saturated or unsaturated, preferably saturated, nonvolatile nonpolar hydrocarbon-based oils.

The linear or branched, nonvolatile nonpolar hydrocarbon-based oil(s) are more particularly compounds comprising only carbon and hydrogen atoms (in other words nonvolatile oils of hydrocarbon type).

Said linear or branched nonpolar oils may be of mineral or synthetic origin, for instance:
liquid paraffin,
squalane,
isocicosane,
mixtures of saturated, linear hydrocarbons, more particularly C₁₅-C₂₈ hydrocarbons, such as the mixtures whose INCI names are, for example, the following: C15-C19 Alkane (INCI name), C18-C21 Alkane (INCI name), C21-C28 Alkane (INCI name), for instance the products Gemseal 40, Gemseal 60 and Gemseal 120 sold by Total, Emogreen L19 sold by SEPPIC
hydrogenated or non-hydrogenated polybutenes, for instance the products of the Indopol range sold by INEOS Oligomers,
hydrogenated or non-hydrogenated polyisobutenes, for instance the nonvolatile compounds of the Parleam® range sold by Nippon Oil Fats,
hydrogenated or non-hydrogenated polydecenes, for instance the nonvolatile compounds of the Puresyn® range sold by ExxonMobil),
and mixtures thereof.

### Nonvolatile Silicone Oils

The nonvolatile oil may also be chosen from phenylated or non-phenylated non-volatile silicone oils. More particularly, said silicone oils are free of (poly)alkoxylated groups, notably such as (poly)ethoxylated or (poly)propoxylated groups, or (poly)glycerolated groups.

For the purposes of the invention, the term “silicone oil” means an oil comprising at least one silicon atom, and notably at least one Si—O group.

More particularly, the phenylated or non-phenylated nonvolatile silicone oil is chosen from dimethicones, trimethylpentaphenyltrisiloxanes, tetramethyltetraphenyltrisiloxanes, diphenyl dimethicones, trimethylsiloxyphenyl dimethicones, phenyl trimethicone and diphenylsiloxyphenyl trimethicone, and also mixtures thereof.

These products are notably sold under the names PH-1555 HRI Cosmetic Fluid (trimethyl pentaphenyl trisiloxane) and Dow Corning 556 Cosmetic Grade Fluid (phenyl trimethicone) by Dow Corning; diphenyl dimethicones such as the products KF-54, KF54HV, KF-50-300CS, KF-53 d and KF-50-100CS or Diphenylsiloxy Phenyl Trimethicone KF56 A sold by Shin-Etsu; the products Belsil PDM 1000 and Belsil PDM 20 sold by Wacker Chemie (trimethylsiloxy phenyl dimethicone), alone or as mixtures. The values in parentheses represent the viscosities at 25° C. (standard ASTM D-445).

More particularly, the concentration of oily phase in the composition, if it is present, is between 1% and 30% by weight, preferably between 1% and 20% by weight, relative to the total weight of said composition.

According to a first variant of the invention, if the oily phase is present in the composition, then it comprises at least one nonvolatile oil. Preferably, according to this variant, the oil(s) are chosen from polar nonvolatile hydrocarbon-based oils. Preferably, the nonvolatile oil is not chosen from nonvolatile silicone oils.

In accordance with another embodiment of the invention, the oily phase is present and comprises at least one volatile oil, preferably hydrocarbon-based volatile oil. More particularly, the content of volatile oil, preferably hydrocarbon-based volatile oil, is less than or equal to 20% by weight, notably less than or equal to 10% by weight, more particularly less than or equal to 9% by weight, preferably less than or equal to 8% by weight and even more precisely less than or equal to 5% by weight, relative to the total weight of the composition. Advantageously, the composition does not comprise any volatile silicone oil.

### Waxes

The composition may optionally comprise at least one wax.

For the purposes of the present invention, the term “wax” means a lipophilic compound, which is solid at 25° C., with a reversible solid/liquid change of state, which has a melting point above or equal to 40° C. that may go up to 120° C.

For the purposes of the invention, the melting point corresponds to the temperature of the most endothermic peak observed in thermal analysis (DSC) as described in the standard ISO 11357-3; 1999. The melting point of the wax may be measured using a differential scanning calorimeter (DSC), for example the calorimeter sold under the name DSC Q2000 by the company TA Instruments.

The measuring protocol is as follows:

A sample of approximately 5 mg of wax is placed in a “hermetic aluminum capsule” crucible.

The sample is subjected to a first temperature rise passing from −20° C. to 120° C., at a heating rate of 10° C./minute, it is then cooled from 120° C. to −20° C. at a cooling rate of 10° C./minute and is finally subjected to a second temperature rise passing from −20° C. to 120° C. at a heating rate of 5° C./minute. During the second temperature rise, the melting point value of the wax is measured, which corresponds to the value of the top of the most endothermic peak observed on the melting curve, representing the variation in the difference in power absorbed as a function of the temperature.

The waxes may be hydrocarbon-based waxes or silicone waxes, and may be of plant, mineral, animal and/or synthetic origin.

More particularly, the waxes are chosen from nonpolar hydrocarbon-based waxes, polar, preferably ester, hydrocarbon-based waxes, and also mixtures thereof.

Among the nonpolar hydrocarbon-based waxes (i.e. waxes comprising only carbon and hydrogen atoms in their structure), mention may notably be made of polyethylene waxes, microcrystalline waxes, paraffin waxes, ozokerite, polymethylene waxes, waxes obtained by Fischer-Tropsch synthesis, microwaxes, notably of polyethylene, and also mixtures thereof.

Among the polar hydrocarbon-based waxes (therefore comprising carbon, hydrogen and oxygen atoms), the waxes advantageously comprising at least one alcohol, ester and/or ether group are suitable.

Waxes that may notably be used include hydrocarbon-based waxes, such as beeswax, lanolin wax; sunflower wax, rice wax, carnauba wax, candelilla wax, ouricury wax, Japan wax, berry wax, shellac wax and sumac wax; montan wax, and also mixtures thereof.

Mention may also be made of waxes obtained by catalytic hydrogenation of animal or plant oils containing linear or branched C₈-C₃₂ fatty chains. Among these waxes, mention may notably be made of hydrogenated jojoba oil, hydrogenated palm oil, hydrogenated sunflower oil, hydrogenated castor oil, hydrogenated coconut kernel oil, hydrogenated lanolin oil and bis(1,1,1-trimethylolpropane) tetrastearate sold under the name Hest 2T-4S® by Heterene, and bis(1,1,1-trimethylolpropane) tetrabehenate sold under the name Hest 2T-4B® by Heterene.

Also suitable are waxes of C₂₀-C₄₀ alkyl (hydroxystearyloxy)stearate type, alone or as a mixture, or a C₂₀-C₄₀ alkyl stearate. Such waxes are especially sold under the names Kester Wax K 82 P®, Hydroxypolyester K 82 P®, Kester Wax K 80 P® and Kester Wax K82H by the company Koster Keunen.

Mention may be made, as alcohol wax, of mixtures of saturated linear C₃₀-C₅₀ alcohols, such as, for example, the wax Performacol 550-L Alcohol from New Phase Technology, stearyl alcohol and cetyl alcohol.

The content of wax(es), if the composition comprises any, varies from 0.1% to 5% by weight, more particularly from 0.5% to 5% by weight, relative to the total weight of the composition.

### Pasty Compounds

The composition may optionally comprise at least one pasty compound.

For the purposes of the present invention, the term “pasty compound” means a lipophilic fatty compound with a reversible solid/liquid change of state, and including, at a temperature of 20° C., a liquid fraction and a solid fraction. Thus, a pasty compound may have a starting melting point below 20° C. Moreover, the pasty compound may have, in the solid state, an anisotropic crystalline organization. The melting point of the pasty fatty substance is determined according to the same principle as that described in detail above for the waxes.

In the case of a pasty compound, the measuring protocol is, however, as follows: A sample of 5 mg of pasty fatty substance placed in a crucible is subjected to a first temperature rise passing from −20° C. to 100° C., at a heating rate of 10° C./minute, it is then cooled from 100° C. to −20° C. at a cooling rate of 10° C./minute and it is finally subjected to a second temperature rise passing from −20° C. to 100° C. at a heating rate of 5° C./minute.

The melting point of the pasty fatty substance is the value of the temperature corresponding to the top of the peak on the curve representing the variation in the difference in power absorbed as a function of the temperature.

It should be noted that the liquid fraction by weight of the pasty fatty substance at room temperature is equal to the ratio of the heat of fusion consumed at room temperature to the heat of fusion of the pasty fatty substance.

The heat of fusion of the pasty fatty substance is the heat consumed by said substance in order to pass from the solid state to the liquid state. The pasty fatty substance is said to be in the solid state when all of its mass is in crystalline solid form. The pasty fatty substance is said to be in the liquid state when all of its mass is in liquid form. The heat of fusion of the pasty fatty substance is the amount of energy required to make the pasty fatty substance change from the solid state to the liquid state. It is expressed in J/g. The heat of fusion of the pasty fatty substance is equal to the arca under the curve of the thermogram obtained.

Preferably, this or these pasty hydrocarbon-based compound(s) are chosen from:
plant butters, for instance mango butter, such as the product sold under the reference Lipex® 203 by the company Aarhuskarlshamn, shea butter, in particular the product whose INCI name is Butyrospermum parkii Butter, such as the product sold under the reference Sheasoft® by the company Aarhuskarlshamn, cupuacu butter (Rain Forest RF3410 from the company Beraca Sabara), murumuru butter (Rain Forest RF3710 from the company Beraca Sabara), cocoa butter; babassu butter such as the product sold under the name Cropure® Babassu by Croda, and also orange wax, for example the product sold under the reference Orange Peel Wax by the company Koster Kounen,
partially hydrogenated plant oils, for instance hydrogenated soybean oil, hydrogenated coconut kernel oil, hydrogenated rapeseed oil, mixtures of hydrogenated plant oils such as the mixture of hydrogenated soybean, coconut kernel, palm and rapeseed plant oil, for example the mixture sold under the reference Akogel® by the company Aarhuskarlshamn (INCI name Hydrogenated Vegetable Oil), the transisomerized partially hydrogenated jojoba oil manufactured or sold by the company Desert Whale under the commercial reference Iso-Jojoba-50®, partially hydrogenated olive oil, for instance the compound sold under the reference Beurrolive by the company Soliance,
esters of hydrogenated castor oil and of C16-C22 fatty acids, in particular of isostearic acid, such as the compound with the INCI name Hydrogenated Castor Oil Isostearate, for example Salacos HCIS (V-L) sold by Nisshin Oil,
triglycerides of optionally (completely or partially) hydrogenated, saturated or unsaturated, linear or branched, optionally monohydroxylated or polyhydroxylated, preferably C12-C18 fatty acids; for instance the glycerides of saturated C12-C18 fatty acids sold under the name Softisan 100® by Cremer Oleo (INCI name: Hydrogenated Coco-Glycerides),
polyesters resulting from the condensation of a linear or branched, C6-C10 dicarboxylic acid and of an ester of diglycerol and of optionally hydroxylated, lincar or branched, C6-C20 monocarboxylic acids, in particular such as the ester obtained by condensation of adipic acid and of a mixture of esters of diglycerol with a mixture of C6-C20 fatty acids, such as caprylic acid, capric acid, stearic acid, isostearic acid and 12-hydroxystearic acid, and having the INCI name Bis-Diglyceryl Polyacyladipate-2. This type of compound is notably sold under the reference Softisan® 649 by Cremer Oleo.
the polyesters obtained from a dimer acid, said acid being unsaturated and comprising from 16 to 24 carbon atoms, and from at least one alcohol or polyol, for instance:
   esters of dimer diol (for example dilinoleyl alcohol) and of dilinoleic acid, the hydroxyl groups of which are esterified by a mixture of phytosterols, of behenyl alcohol and of isostearyl alcohol, for example the ester sold under the name Plandool G by Nippon Fine Chemical (INCI name: Bis-Behenyl/Isostearyl/Phytosteryl Dimer Dilinoleyl Dimer Dilinoleate);
   esters of dilinoleic acid and of a mixture of phytosterols, of isostearyl alcohol, of cetyl alcohol, of stearyl alcohol and of behenyl alcohol, for example the ester sold under the name Plandool H or Plandool S by Nippon Fine Chemical (INCI name: Phytosteryl/Isostearyl/Cetyl/Stearyl/Behenyl Dimer Dilinoleate);
   esters of hydrogenated castor oil and of dilinoleic acid, such as those sold under the names Risocast-DA-L or Risocast-DA-H by Kokyu Alcohol Kogyo (INCI name: Hydrogenated Castor Oil Dimer Dilinoleate).

Preferably, if the composition comprises any, the pasty hydrocarbon-based compound(s) are chosen from plant butters, partially hydrogenated plant oils, the compounds with the INCI name Phytosteryl/Isostearyl/Cetyl/Stearyl/Behenyl Dimer Dilinoleate, Hydrogenated Coco-Glycerides, and Bis-Diglyceryl Polyacyladipate-2 and also the mixtures thereof.

If the composition comprises at least one pasty hydrocarbon-based compound, its/their content is more particularly less than or equal to 20% by weight, preferably between 0.5% and 10% by weight, with respect to the total weight of the composition.

### Cosmetic Additives

The composition of the invention may contain additives that are common in cosmetics. Mention may notably be made of antioxidants, preserving agents, neutralizers, gelling agents or thickeners, surfactants, cosmetic active agents, for instance emollients, moisturizers or vitamins, and mixtures thereof.

### Antioxidants

In particular, the antioxidants are used to prevent the oxidation of the polyphenol X. They may be chosen from sulfites and metabisulfite, and reducing agents of thiol type, in particular cysteine. Mention may also be made of carotenes and lycopenes, which also act as liposoluble dyes.

These additives may be present in compositions (A), (B), (C) and/or (D) in a content ranging from 0.01% to 15.0% relative to the total weight of the composition.

Needless to say, those skilled in the art will take care to select the optional additional additives and/or the amount thereof such that the advantageous properties of the composition according to the invention are not, or are not substantially, adversely affected by the envisaged addition.

Compositions (A), (B), (C) and/or (D) may be manufactured via the known processes, generally used in the cosmetic field.

Compositions (A), (B), (C) and/or (D) used according to the invention may be care products, makeup products or hybrid products (with a care and makeup capacity) for keratin materials such as the skin, the eye contour or the lips.

### Makeup and/or Care Methods

Another subject of the invention is a cosmetic method for making up and/or caring for the skin and/or the lips, preferably the lips, according to the invention, consisting in applying the composition as defined above to the skin and/or the lips.

According to another variant, the method may involve a composition (M).

Thus, another method for making up the skin and/or the lips, preferably the lips, consists in successively applying:
a) a layer constituted of at least one composition as defined above, and
b) a layer constituted of at least one composition (M) comprising at least one oil.

It would not be excluded to reverse the order of application of the two compositions.

One and/or the other of the compositions may comprise at least one colorant; preferably, the composition applied during the first step comprises at least one colorant.

The application of the compositions is advantageously carried out without mixing. More particularly, it is preferred for the composition of the second step to be applied once the composition of the first step has at least partially dried. By way of indication, the second step is carried out a few minutes after the first.

### Composition (M)

The composition (M) comprises at least one oil. Reference could be made to the description of the oils indicated previously.

The composition (M) also optionally comprises at least one colorant chosen from those defined previously.

The composition (M) may advantageously comprise at least one silicone or nonsilicone film-forming polymer, or a mixture.

As examples of silicone film-forming polymers, mention may notably be made of silicone resins, such as the resins whose INCI names are the following: trimethylsiloxysilicate, phenylpropyldimethylsiloxysilicate, polypropylsilsesquioxane, polymethylsilsesquioxane, and also the mixtures thereof. Also suitable are the polymers having the following INCI names: Acrylates/Polytrimethylsiloxymethacrylate Copolymer, Acrylates/Dimethicone, and also the mixtures thereof.

The film-forming polymer may also be chosen from ethyl cellulose, or ethylenic block copolymers, for instance Acrylic Acid/Isobutyl Acrylate/Isobornyl Acrylate Copolymer (INCI name).

If the composition (M) comprises any, the content of film-forming polymer(s) advantageously varies between 0.1% and 50% by weight, more particularly from 1% to 30% by weight, relative to the total weight of the composition (M).

The composition (M) may also contain one or more ingredients commonly used in this type of product, such as pasty compounds, in particular hydrocarbon-based pasty compounds, for instance compounds such as plant butters (for example shea butter), ester compounds derived from dimer diol dimer dilinoleate (for example bis-behenyl/isostearyl/phytosteryl dimer dilinoleyl dimer dilinoleate) or else bis-diglyceryl polyacyladipate-2; waxes, notably hydrocarbon-based waxes, which are polar (for example of ester type) or nonpolar (for example polyethylene wax, microscrystalline wax, etc.); organic solvents; thickeners; colorless or white fillers, which may be lamellar, spherical or in the form of fibers; cosmetic active agents such as vitamins, UV sunscreens, moisturizers and antioxidants.

The composition (M) may optionally comprise water.

The composition (M) may be a product, notably for making up and/or caring for the skin, the face, the cheeks or the eye contour. If these compositions (M) are intended for makeup, they then contain at least one colorant as defined previously.

The compositions (M) for the skin are preferably gels, creams, milks or lotions. They may be aqueous dispersions, oily anhydrous compositions or multi-phase compositions such as oil-in-water emulsions, water-in-oil emulsions, or two-phase or three-phase compositions.

The compositions (M) for the skin may also be in hot-poured solid form.

The composition (M) may be a product intended to be applied to the lips, notably a lipstick, a lip gloss or a lip balm.

The compositions (M) for the lips may be in solid form such as a stick, or products in a dish or else in liquid form.

They may be anhydrous or aqueous, notably water-in-oil or oil-in-water emulsions.

### Packaging and Applicators

Compositions (A), (B), (C), (D) and/or (M) according to the invention may each be packaged in a container delimiting at least one compartment that comprises said composition, said container being closed by a closing member.

The container may be in any suitable form. It may notably be in the form of a bottle, a tube, a jar or a case.

The closing member may be in the form of a removable stopper, a lid or a cover, notably of the type including a body fixed to the container and a cap articulated on the body. It may also be in the form of a member for selectively closing the container, notably a pump, a valve or a flap valve.

The container may be combined with an applicator, for example in the form of a fine brush (as described, for example, in patent FR 2 722 380), in the form of a deformable or non-deformable member, made of foam or of elastomer, and which is optionally flocked. The applicator may also be free (sponge) or securely fastened to a stem borne by the closing member, as described, for example, in patent U.S. Pat. No. 5,492,426. The applicator may be securely fastened to the container, as described, for example, in patent FR 2 761 959.

The product may be contained directly in the container, or indirectly.

The closing member may be coupled to the container by screwing. Alternatively, the coupling between the closing member and the container occurs other than by screwing, notably via a bayonet mechanism, by click-fastening or by gripping. The term “click-fastening” in particular means any system involving the crossing of a bead or cord of material by elastic deformation of a portion, notably of the closing member, followed by return to the clastically unconstrained position of said portion after the bead or cord has been crossed.

The container may be at least partially made of thermoplastic material. Examples of thermoplastic materials that may be mentioned include polypropylene and polyethylenc.

The container may have rigid or deformable walls, notably in the form of a tube or a tube bottle.

The container may comprise means intended to bring about or facilitate the dispensing of the composition. By way of example, the container may have deformable walls so as to make the composition exit in response to excess pressure inside the container, which excess pressure is brought about by the elastic (or non-elastic) squeezing of the walls of the container.

The container may be equipped with a drainer positioned in the vicinity of the opening of the container. Such a drainer makes it possible to wipe the applicator and possibly the stem to which it may be securely fastened. Such a drainer is described, for example, in patent FR 2 792 618.

Throughout the description, including the claims, the expression “comprising a” should be understood as being synonymous with “comprising at least one”, unless otherwise specified.

The expressions “between . . . and . . . ” and “ranging from . . . to . . . ” should be understood as meaning limits included, unless otherwise specified.

In addition, the sum of the amounts of the ingredients of the composition represents 100% by weight of the composition.

The invention is illustrated in greater detail by the examples presented below.

Unless otherwise indicated, the amounts indicated are expressed as mass percentages.

The examples which follow are presented by way of illustration and without limitation of the invention.

### EXAMPLES

### Example 1

The following compositions were prepared:

TABLE 1

| | Composi- | Composi- |
|---|---|---|
| Ingredients (INCI name or chemical name) | tion 1 | tion A |
| Tannic acid | 5 | 5 |
| (Brewtan F - Ajinomoto Omnichem NV) | | |
| Polyglyceryl-6 Dicaprate | 7.5 | 7.5 |
| (Sunsoft Q-102H-C - Taiyo Kagaku) | | |
| Erythorbic acid | 0.5 | |
| Ethanol | qs 100 | qs 100 |

### Preparation Process

At low temperature, mix the tannic acid, ethanol, and also erythorbic acid, where appropriate, using a Rayneri blender.

Next, introduce the polyglycerolated compound once the tannic acid and erythorbic acid have dissolved.

### Evaluation

The concentration of ethyl gallate and gallic acid is measured for each composition (composition 1 according to the invention and comparative composition A).

Next, two samples of each composition are prepared in order to subject them to the following two processes:
(1) storage for 1 month at room temperature (25° C., in the absence of light)
(2) storage for 1 month at 45° C. (in an oven, in the absence of light). The assay is carried out once the composition has returned to room temperature.

### Protocol for Measuring Ethyl Gallate And Gallic Acid

HPLC chromatography on a RESTEK Raptor Biphenyl 2.6 μm 2.1×100 mm column Linear elution gradient Eluent A→Eluent B:

Eluent A: CH₃ CN/H₂O/H₃PO₄ (85%): 1/99/0.1% (volume).

Eluent B: CH₃ CN/H₂O/CH₃OH/H₃PO₄ (85%): 1/9/90/0.1% (volume).

Sample dissolved with an H₂O/DMSO mixture (50/50% by volume)

### Results

TABLE 2

| | Composition 1 | Composition A |
|---|---|---|
| T0 | | |
| Ethyl gallate | 0.000% | 0.002% |
| Gallic acid | 0.003% | 0.003% |
| 1 month at room T | | |
| Ethyl gallate | 0.01% | 0.33% |
| Gallic acid | 0.004% | 0.006% |
| 1 month at T 45° C. | | |
| Ethyl gallate | 0.04% | 1.79% |
| Gallic acid | 0.005% | 0.017% |

It is observed that the presence of erythorbic acid makes it possible to significantly reduce the content of ethyl gallate and gallic acid over time.

### Makeup Evaluation

On making up the lips, each composition is applied very easily with a dip applicator, making it possible to obtain a very thin film on the lips.

A very comfortable, non-tacky deposit was obtained.

The assembly does not transfer and has a good resistance with respect to water and oil (protocol described in detail below).

### Example 2

The following compositions were prepared.

TABLE 3

| | Composi- | Composi- |
|---|---|---|
| Ingredients (INCI name or chemical name) | tion 2 | tion B |
| Tannic Acid | 5 | 5 |
| (Brewtan F - Ajinomoto Omnichem NV) | | |
| Polyglyceryl-6 Dicaprate | 7.5 | 7.5 |
| (Sunsoft Q-102H-C - Taiyo Kagaku) | | |
| Erythorbic Acid | 0.5 | — |
| Red 7 | 10 | 10 |
| Ethanol | qs 100 | qs 100 |

### Preparation Method

At low temperature, mix the tannic acid and ethanol, where appropriate with the erythorbic acid, using a Rayneri blender.

Next, introduce the polyglycerolated compound once the tannic acid and erythorbic acid have dissolved.

Once the mixture has been homogenized, sprinkle in the pigment while stirring and disperse for 15 minutes.

### Evaluation

The concentration of ethyl gallate and gallic acid is measured for each composition according to the protocol described in detail in example 1.

### Results

TABLE 4

| | Composition 2 | Composition B |
|---|---|---|
| T0 | | |
| Ethyl gallate | 0.000% | 0.001% |
| Gallic acid | 0.004% | 0.004% |
| 1 month at room T | | |
| Ethyl gallate | 0.01% | 0.10% |
| Gallic acid | 0.004% | 0.005% |
| 1 month at T 45° C. | | |
| Ethyl gallate | 0.04% | 1.79% |
| Gallic acid | 0.005% | 0.017% |

It is observed that the presence of erythorbic acid makes it possible to significantly reduce the content of ethyl gallate and gallic acid over time.

### Makeup Evaluation

On making up the lips, each composition is applied very easily with a dip applicator, making it possible to obtain a very thin film on the lips, of a vivid, uniform color.

A very comfortable, non-tacky deposit was obtained.

The assembly does not transfer and has a good resistance with respect to water and oil (protocol described in detail below).

### Example 3

The following compositions were prepared:

TABLE 5

| | Composi- | Composi- |
|---|---|---|
| Ingredients (INCI name or chemical name) | tion 3 | tion C |
| Tannic acid | 15 | 15 |
| (Brewtan F - Ajinomoto Omnichem NV) | | |
| Polyglyceryl-6 Dicaprate | 5 | 5 |
| (Sunsoft Q-102H-C - Taiyo Kagaku) | | |
| Polyglyceryl-4 caprate | 5 | 5 |
| Polyglyceryl-5 laurate | 5 | 5 |
| Phytic acid | 3 | 0 |
| (Tsuno Rice Fine Chemicals Phytic Acid (IP6), | | |
| Tsuno Rice Fine Chemicals) | | |
| Red 7 | 10 | 10 |
| Ethanol | qs 100 | qs 100 |

### Preparation Method

At low temperature, mix the tannic acid, ethanol, and also phytic acid, where appropriate, using a Rayneri blender. Next, introduce the polyglycerolated compounds once the tannic acid and phytic acid have dissolved. Sprinkle in the Red 7 pigment and leave stirring for 15 minutes.

### Evaluation

Each composition is stored for 15 days at 45° C. (in an oven, in the absence of light). Then the assay is carried out for each composition, once the composition has returned to room temperature.

### Protocol for Measuring The Ethyl Gallate And Gallic Acid:

HPLC chromatography on a RESTEK Raptor Biphenyl 2.6 μm 2.1×100 mm column

Linear elution gradient Eluent A→Eluent B:

Eluent A: CH₃CN/H₂O/H₃PO₄ (85%): 1/99/0.1% (volume).

Eluent B: CH₃CN/H₂O/CH₃OH/H₃PO₄ (85%): 1/9/90/0.1% (volume).

Sample dissolved with an H₂O/DMSO mixture (50/50% by volume)

### Results

TABLE 6

| | Composition 3 | Composition C |
|---|---|---|
| Ethyl gallate | 0.03% | 1.82% |
| Gallic acid | 0.0178% | 0.04% |

It is observed that the presence of phytic acid makes it possible to significantly reduce the content of ethyl gallate and gallic acid over time.

### Makeup Evaluation

On making up the lips, each composition is applied very easily with a dip applicator, making it possible to obtain a very thin film on the lips.

The assembly does not transfer and has a good resistance with respect to water and oil (described in detail below).

### Protocol for Evaluating the Performances

### 1. Test Preparation:

Support: Beige Supplale (2.5×5 cm) (sold by Soudotique).

Spread composition (D) over the entire surface 3 times running in order to have a uniform deposit. Repeat the operation on two other strips.

Leave the deposit to dry on a plate heated at 34° C. for 30 minutes.

Optionally take a photo of each support with the (made-up) deposit before the stress.

### 2. Stresses:

### Preparation of a Tissue for Each Stress:

Fold each tissue twice on the long edge then twice in the other direction to form a square.

### Dry Resistance:

Rub one of the three made-up supports lengthwise once with the folded tissue; the force applied is that normally exerted when removing make up from the skin or the lips.

Observe the state of the rubbed support and also the used surface of the tissue, in particular the remaining color and the transferred color.

Optionally take a photo.

Should several passes be carried out, they would then be carried out with the same force and always in the same direction (i.e. after each pass, the tissue is lifted in order to be repositioned at the “start” of the strip so as to be reapplied on the deposit in the same way as in the preceding pass). Optionally take photo between each step or only at the end of the evaluation. This this type of process can be carried out in order to evaluate the overall resistance of the deposit.

### Water Resistance:

Insert the second made-up support, without folding it, into a centrifuge tube.

Add 10 grams of demineralized water.

Centrifuge for 10 minutes at 450 g.

Optionally take a photo of the support after mixing, immediately after the operation.

Rub once with a tissue along the length of the support, without waiting, with the same force as that applied for the dry resistance.

Observe the state of the rubbed support and also the used surface of the tissue, in particular the remaining color and the transferred color.

Optionally take a photo.

The protocol for several passes is the same as that described in detail above for the dry resistance.

### Oil Resistance:

Carry out the same protocol as for the water resistance, on the third made-up support, replacing the water with the same amount of olive oil (Refined Olive Oil—Aarhuskarlshamn).

### Grading:

For each stress, grade the result according to the table below:

TABLE 7

| | | Surface of the fabric in |
|---|---|---|
| Grade | State of the deposit | contact with the deposit |
| −− | Complete or partial elimination | Very intense coloration - |
| | of the deposit on the rubbed zone; | very significant to complete |
| | the surface of the support appears | transfer of the color |
| | in places | |
| − | Partial elimination resulting in a | Intense coloration - |
| | significantly and visibly less | significant transfer of the |
| | intense coloration of the deposit | color |
| + | Reduction in the intensity of the | Medium coloration - |
| | color of the deposit is perceptible | medium transfer of the |
| | but does not reveal the support | color |
| ++ | No substantial variation in the | Slight coloration - little |
| | color of the deposit | transfer of the color |
| +++ | No variation in the color of the | No coloration or coloration |
| | deposit | that is barely visible - no to |
| | | very little transfer of the |
| | | color |

It should be noted that to facilitate the evaluation of the wear property of the deposit, this deposit comprises at least one colorant having a sufficiently intense coloration (for example a red colorant, such as Red 7 notably).

In the case of comparing several compositions, the latter have the same color (same amount and same colorant).

It should be noted that the wear property of the deposit, and also other properties (homogeneity, comfort, etc.) can also be evaluated by a Sensory Panel (group of trained experts enabling a description of technical characteristics to be obtained—standards ISO 8586, ISO 11132, ISO 13299).

1. A composition for making up and/or caring for the skin and/or the lips, preferably the lips, more particularly a make-up composition, comprising
a) at least one polyphenol X comprising at least two different phenol groups;
b) at least one compound Y chosen from those capable of forming at least two hydrogen bonds with said phenol groups of the polyphenol X, which is preferably nonionic, polyoxyethylenated and/or poly-oxypropylenated and/or (poly)glycerolated;
c) at least one C2-C8, preferably C2-C5, monoalcohol, preferably ethanol;
d) at least one acid chosen from phytic acid, acetic acid, at least one hydroxylated carboxylic acid chosen from C6 aromatic or C2-C10 non-aromatic monocarboxylic or polycarboxylic acids, bearing at least one OH radical in the alpha or beta position of a carboxylic group that is free or engaged in a heterocycle; ferulic acid; and also mixtures thereof.

2. The composition as claimed in claim 1, characterized in that the polyphenol X is chosen from catechin tannins, notably chosen from gallotannins and ellagitannins.

3. The composition as claimed in either one of claims 1 and 2, characterized in that the polyphenol X is epigallocatechin, notably a green tea extract, in particular comprising at least 45% by weight of epigallocatechin relative to the weight of said extract.

4. The composition as claimed in claim 1, characterized in that the polyphenol X is a procyanidin or a mixture of procyanidins, in particular an extract of maritime pine bark, notably comprising at least 65% by weight of procyanidins relative to the total weight of said extract.

5. The composition as claimed in either one of claims 1 and 2, characterized in that the polyphenol X is tannic acid.

6. The composition as claimed in any one of the preceding claims, characterized in that the content of polyphenol X is at least 2% by weight, preferably between 2% and 30% by weight, and even more particularly from 3% to 25% by weight, relative to the total weight of said composition.

7. The composition as claimed in any one of the preceding claims, characterized in that the compounds Y have a molar mass of greater than 200 g/mol, more particularly greater than 350 g/mol.

8. The composition as claimed in any one of the preceding claims, characterized in that the compound(s) Y is/are chosen from polyoxyethylenated and/or polyoxypropylenated and/or polyglycerolated linear, branched or crosslinked silicone compounds, polyoxyethylenated and/or polyoxypropylenated alcohols, polyoxyethylenated and/or polyoxypropylenated alkylglycols or glycerol, polyethylene glycols, Poloxamers, polyoxyethylenated and/or polyoxypropylenated and/or (poly)glycerolated esters, polyoxyethylenated esters of sorbitol or sorbitan and polysorbates, polyoxyethylenated sugar ester or ether derivatives, polyoxyethylenated alkylamines, and also mixtures thereof.

9. The composition as claimed in any one of the preceding claims, characterized in that the compound(s) Y is/are chosen from:
polyoxyethylenated fatty alcohols of R(O—CH₂—CH₂)ₒ—OH type, R representing a C₈-C₃₀ alkyl radical, o representing an average integer ranging from 2 to 50;
polyoxyethylenated and/or oxypropylenated alcohols of the following type: R—(O—C(CH₃)H—CH₂)ₒ—(O—CH₂—CH₂)ₚ—OH; R representing a C₄-C₃₀ alkyl radical, o and p, independently of one another, representing an average integer ranging from 1 to 50;
esters of polyglycerol comprising 2 to 20 glycerol units and of saturated or unsaturated carboxylic acid(s) or carboxylic acid polymer(s) comprising from 6 to 40 carbon atoms; or derivatives of plant oils;
esters of glycerol and of saturated or unsaturated C₆-C₄₀, more particularly C₈-C₃₀, carboxylic acid(s) or carboxylic acid polymer(s) or of derivatives of plant oils, said esters being polyoxyethylenated, comprising from 2 to 200 oxyethylene units;
polysorbates;
mixtures thereof.

10. The composition as claimed in any one of the preceding claims, characterized in that the compounds Y are chosen from polyglyceryl esters chosen from the following compounds, denoted by their INCI name: Polyglyceryl-2 Stearate, Polyglyceryl-2 Diisostearate, Polyglyceryl-3 Diisostearate, Polyglyceryl-3 Dicitrate/Stearate, Polyglyceryl-4 Diisostearate, Polyglyceryl-4 Caprate, Polyglyceryl-5 Laurate, Poylglyceryl-5 Oleate, Polyglyceryl-6 Caprylate, Polyglyceryl-6 Dicaprate, Polyglyceryl-6 Distearate, Polyglyceryl-6 Caprylate/Caprate, Polyglyceryl-6 Dioleate, Polyglyceryl-6 Trilaurate, Polyglyceryl-10 Laurate, Polyglyceryl-10 Dioleate, polyglycerolated apricot kernel oil esters comprising from 3 to 10 glycerol units, and also the mixtures thereof.

11. The composition as claimed in any one of the preceding claims, characterized in that the content of compound Y represents at least 1% by weight, more particularly at least 2% by weight, preferably from 2% to 40% by weight and even more preferentially from 3% to 35% by weight, relative to the total weight of said composition.

12. The composition as claimed in any one of the preceding claims, characterized in that the ratio of the mass of polyphenol(s) X, expressed as active material, to the mass of the compounds Y, expressed as active material, varies between 0.25 and 3, preferably between 0.5 and 2.

13. The composition as claimed in any one of the preceding claims, characterized in that the content of C2-Cx monoalcohol is between 25% and 98% by weight, in particular between 30% and 90% by weight and preferably between 35% and 85% by weight, relative to the total weight of the composition.

14. The composition as claimed in any one of the preceding claims, characterized in that the acid is a hydroxylated carboxylic acid chosen from C₂-C₁₀ non-aromatic monocarboxylic or polycarboxylic acids bearing at least one free OH radical in the alpha position of a carboxylic acid group that is free or engaged in a heterocycle; the hydroxylated carboxylic acid comprising a carboxylic acid group engaged in a hetcrocycle or comprising 1 to 4 free carboxylic acid groups; the number of free hydroxyl radicals varying from 1 to 4; it being possible for said hydroxylated carboxylic acid to optionally comprise an ester function.

15. The composition as claimed in any one of the preceding claims, characterized in that the acid is a hydroxylated carboxylic acid chosen from alpha-hydroxy acids such as in particular erythorbic acid, ascorbic acid, citric acid, lactic acid, and also isomers thereof; glycolic acid; ascorbyl palmitate; ferulic acid; salicylic acid; and also mixtures thereof; and preferably from erythorbic acid, ascorbic acid, citric acid, lactic acid, and also isomers thereof; glycolic acid, and also mixtures thereof.

16. The composition as claimed in any one of the preceding claims, characterized in that the content of hydroxylated carboxylic acid varies from 0.1% to 6% by weight, more particularly from 0.2% to 5% by weight, relative to the total weight of the composition.

17. The composition as claimed in any one of the preceding claims, characterized in that the content of phytic acid and/or of acetic acid varies from 0.1% to 6% by weight, more particularly from 0.2% to 5% by weight, relative to the total weight of the composition.

18. The composition as claimed in any one of the preceding claims, characterized in that it comprises at least water at a content of less than 10% by weight, preferably less than or equal to 5% by weight, relative to the total weight of the composition.

19. The composition as claimed in any one of the preceding claims, characterized in that it comprises at least one colorant, more particularly chosen from colorants that are synthetic, natural or of natural origin; in particular chosen from coated or uncoated pigments, water-soluble dyes, liposoluble dyes, and mixtures thereof.

20. The composition as claimed in any one of the preceding claims, characterized in that it comprises an oily phase, more particularly at a content of between 1% and 30% by weight, preferably between 1% and 20% by weight, relative to the total weight of said composition.

21. The composition as claimed in any one of the preceding claims, characterized in that the composition comprises an oily phase comprising at least one volatile hydrocarbon-based oil, at a content of less than or equal to 20% by weight, more particularly less than or equal to 10% by weight, relative to the total weight of the composition.

22. A method for making up and/or caring for the skin and/or the lips, in particular the lips, in which the composition as claimed in any one of the preceding claims is applied.
